(19) United States
(12) Patent Application Publication
(10) Pub. No.: US 2012/0253838 A1
(43) Pub. Date: Oct. 4, 2012

(54) EXAMINATION RESERVATION MANAGEMENT SYSTEM

(75) Inventors: Hiroshizu Morishima, Utsunomiya-shi (JP); Akihiko Yoshida, Ota-ku (JP); Takafumi Yoshida, Yokohama-shi (JP); Maki Minakuchi, Kawasaki-shi (JP)

(73) Assignees: TOSHIBA MEDICAL SYSTEMS CORPORATION, Otawara-shi (JP); KABUSHIKI KAISHA TOSHIBA, Tokyo (JP)

(21) Appl. No.: 13/386,054

(22) PCT Filed: Oct. 24, 2011
(86) PCT No.: PCT/JP11/05931

§ 371 Date: Jan. 20, 2012

(30) Foreign Application Priority Data
Oct. 25, 2010 (JP) 2010-238761

(51) Int. Cl. G06Q 50/22

(52) U.S. Cl. 705/2

(57) ABSTRACT

An examination reservation management system includes an information acquiring means for acquiring examination reservation information including priority information indicating priority of an examination; a reservation status presenting means for distinguishing and displaying a time frame in which examination can be reserved and a reserved time frame based on the examination reservation information; a reservation input means for making a new examination reservation; a priority information determining means for determining whether or not the new examination reservation can be reserved using the priority information; and a reservation designing means for confirming the examination reservation from the determination result by the priority information determining means.

### TECHNICAL FIELD

Embodiments of the present invention relate to an examination reservation management system used for reservation management of an examination room or an examination device.

### RELATED APPLICATION

This application is based on the benefit of the priority on Japanese Patent Application No. 2010-238761 filed in Japan on Oct. 25, 2010, the entire contents of which are hereby incorporated by reference.

### BACKGROUND ART

In a conventional examination reservation management system, the examination room and the examination device are generally reserved using a time table in which the examination time is time divided.

The work information of doctors and examination technicians in the hospital are also managed, so that such work information can also be referenced. For instance, the examination plan can be ex-post drawn out according to the examination scheduled in advance and the schedule of the doctor, as well as the state of the examination device.

However, in the prior art described above, a problem in that the device can be reserved even if the technician who is to carry out the X-ray photography is not present and the photography cannot be performed, and a problem in that the examination can be reserved even if the examination device is under maintenance or is broken as such information is not interlinked.

When a patient is planning to continuously perform a plurality of examinations, the examination device or the examination room of the examination to be performed next becomes empty if one examination is delayed, which may affect the reservation of other patients and cause waiting time. Furthermore, if the scheduled patient does not come to the hospital, the scheduled examination cannot be performed at the scheduled time band, and thus a time in which the examination device is not used produces. The schedule of the examination technician also becomes free.

Such free time lowers the operating rate of the entire hospital, and becomes a great problem in introducing and maintaining a very expensive latest medical examination apparatus.

### CITATION LIST

### Patent Literature

PLT 1: Japanese Patent Application Laid-Open No. 2006-75512

### SUMMARY OF THE INVENTION

### Technical Problem

An object to be achieved by the present invention is to reduce the free time, and an examination reservation management system enabling an efficient operation is provided.

### Solution to Problem

To achieve the above object, an examination reservation management system of the embodiment includes an information acquiring means for acquiring examination reservation information including priority information indicating priority of an examination; a reservation status presenting means for distinguishing and displaying a time frame in which examination can be reserved and a reserved time frame based on the examination reservation information; a reservation input means for making a new examination reservation; a priority information determining means for determining whether or not the new examination reservation can be reserved using the priority information; and a reservation designing means for confirming the examination reservation from the determination result by the priority information determining means.

### Advantageous Effects of Invention

According to the examination reservation management system of the embodiment, an efficient operation can be carried out for the examination reservation.

### BRIEF DESCRIPTION OF THE DRAWINGS

FIG. 1 is an overall configuration diagram of an examination reservation management system according to a first embodiment.

FIG. 2 is a view showing a configuration of a reservation designing unit of the reservation management system according to the embodiment.

FIG. 3 is a view showing a basic table saved in an examination device database of the examination reservation management system according to the embodiment, where (a) shows a relationship of the examination device and the examination ID, (b) shows the relationship of the examination device and the maintenance information, and (c) shows the relationship of the examination device and the operator.

FIG. 4 is a view showing a basic table saved in an examination room database of the examination reservation management system according to the embodiment.

FIG. 5 is a view showing a basic table saved in a work management database of the examination reservation management system according to the embodiment, where (a) shows the daily work management information of the current day, (b) shows the clock in time presence/absence management information, and (c) shows the employee information.

FIG. 6 is a view showing a basic table saved in a reservation management database of the examination reservation management system according to the embodiment.

FIG. 7 is a view showing a reservation status list of the examination reservation management system according to the embodiment.

FIG. 8 is a view showing a submenu of the examination reservation management system according to the embodiment, where (a) is a display submenu, (b) is a register submenu, and (c) is an automatic change submenu.

FIG. 9 is a flowchart of examination reservation registration of the examination reservation management system according to the embodiment.

FIG. 10 is a view showing a flowchart of reservation automatic change when the operator is absent in the examination reservation management system according to the embodiment.

FIG. 11 is a view showing a flowchart of reservation automatic change when the patient is absent in the examination reservation management system according to the embodiment.

FIG. 12 is a view showing a flowchart of reservation automatic change when the examination device breaks down in the examination reservation management system according to the embodiment.

FIG. 13 is a view showing a flowchart of reservation automatic change when the examination delay occurred in the examination reservation management system according to the embodiment.

FIG. 14 is an overall configuration diagram of an examination reservation management system according to a second embodiment.

FIG. 15 is a view showing a basic table saved in a medicine information database of the examination reservation management system according to the embodiment, where (a) shows a relationship of the medicine necessary for every type of examination, and (b) shows the stock of each medicine.

### DESCRIPTION OF EMBODIMENTS

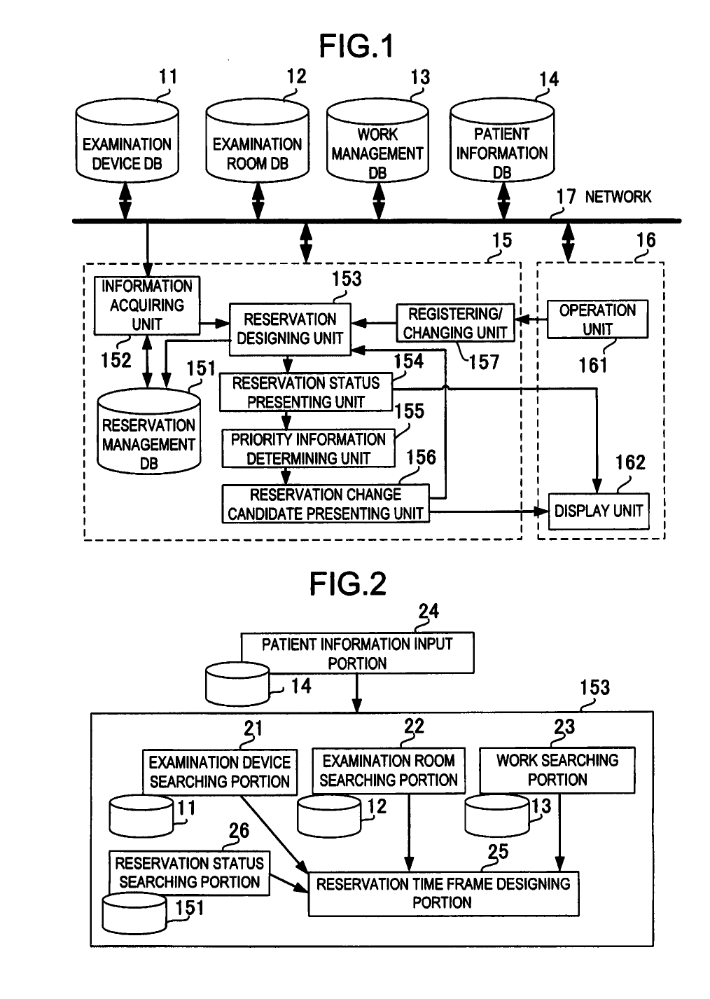

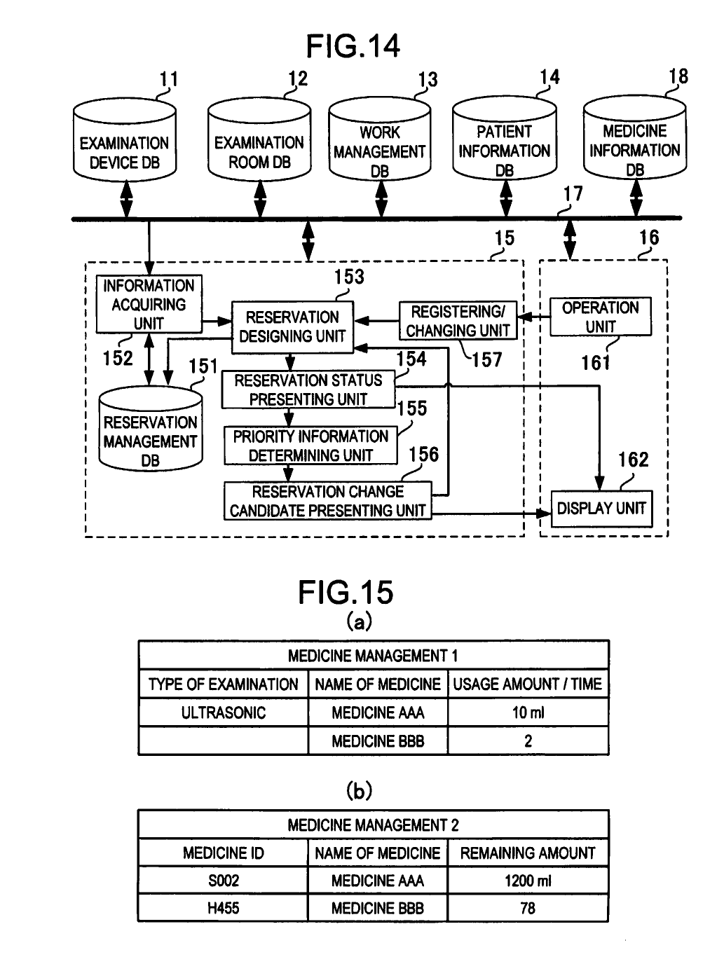

Embodiments for implementing the invention will be hereinafter described in detail with reference to the drawings shown in FIG. 1 to FIG. 15.

### First Embodiment

FIG. 1 is an overall configuration diagram of an examination reservation management system according to one embodiment of the present invention. The reservation management system in one embodiment of the present invention is configured by an examination device database 11 in which examination device information are saved, an examination room database 12 in which examination room information are saved, a work management database 13 in which service information of an operator who operates the examination device such as doctors and examination technicians or even nurses in some cases, a patient information database 14 in which examination information of a patient is saved, a reservation management server 15 for carrying out reservation management from the above information, and a reservation terminal 16 for carrying out display of the examination reservation information, reservation registration input, and the like.

The examination device database 11, the examination room database 12, the work management database 13, the patient information database 14, the reservation management server 15, and the reservation terminal 16 are normally connected to a LAN (Local Area Network) 17 in the hospital, so that information can be mutually exchanged.

The examination device database 11 saves data related to the examination device such as the usage schedule and operation status including maintenance information of the examination device, correspondence relationship with the operator who is authorized to operate each examination device, and the like.

The examination room database 12 saves data related to the examination room such as the usage schedule and operation status including maintenance and cleaning information of the examination room, the correspondence with the examination device that can be used in each examination room, the correspondence with the stock of examination medicines used in the examination, and the like.

The work management database 13 saves data such as the work schedule, the service status of the current day, and the like of the operator authorized to operate each examination device.

The patient information database 14 saves data such as section information (hereinafter referred to as priority information) indicating the priority of the examination reservation including an in/out section indicating whether an admitted patient or an outpatient, an emergency section representing the degree of emergency of the patient, and a changing section representing the changing level of reservation, in addition to the name of disease of the patient, the site to be examined, and the type of examination device.

The reservation management server 15 is configured by an information acquiring unit 152 for acquiring information of the examination device database 11, the examination room database 12, the work management database 13, the patient information database 14, and a reservation management database 151 in which the examination reservation information are saved; a reservation designing unit 153 for designing a time frame in which the examination can be reserved and a time frame in which examination is already reserved based on the information of the information acquiring unit 152; a reservation status presenting unit 154 for presenting the reservation status designed by the reservation designing unit 153 to the reservation terminal 16 in the form of a time table; a priority information determining unit 155 for comparing the priority information associated with the time frame and determining the possibility for changing reservation, or the like; a reservation candidate presenting unit 156 for causing the reservation terminal 16 to display the candidate of the reservation time frame; and a registering/changing unit 157 for receiving registration/change of reservation from the reservation terminal 16.

The reservation terminal 16 is configured by an operation unit 161 with a user interface such as a mouse, and a display unit 162 connected with a monitor or the like for displaying the reservation status.

FIG. 2 is a view showing a configuration example of the reservation designing unit 153 in FIG. 1. The reservation designing unit 153 is configured by an examination device searching portion 21 for searching the available time of the examination device, an examination room searching portion 22 for searching the available time of the examination room, a work searching portion 23 for searching the work information of the operator of the examination device to use in the examination, a patient information input portion 24, a reservation time frame designing portion 25 for designing the reservation time frame.

The operation of the reservation designing unit 153 shown in FIG. 2 will be described. For instance, the patient information such as the patient name and the name of the disease of the patient, the examination information such as the site to examine and the type of examination device, and the priority information such as the in/out section indicating whether the admitted patient or the outpatient, the emergency section representing the degree of emergency of the patient, or the changing section representing the changing level of reservation are input through the information acquiring unit 152 from the patient information database 14 to which patient information such as the in-hospital network HIS (Hospital Information System) is input. With such input, the information acquiring unit 152 acquires the examination device information from the examination device database 11 for reservation designing, and the examination device searching portion 21 searches for the available time frame of the examination device.

The examination room information is also acquired from the examination room database 12, and the available time frame of the examination room is searched by the examination room searching portion 22. Moreover, the work searching portion 23 searches for the operator who can be assigned. The reservation status searching portion 26 searches for the current examination reservation status from the reservation management database 151. According to such information, the reservation time frame designing portion 25 carries out operations such as designing of the available time frame in which reservation can be made, registration of the examination reservation, and re-designing for change of reservation.

After the available time frame in which reservation can be made is designed, a time table in which the time frame in which the examination reservation can be made and the time frame in which the examination is already reserved are distinguished is displayed on a monitor connected to the display unit 162 of the reservation terminal 16 through the reservation status presenting unit 154, and the reservation is registered using the user interface such as the mouse connected to the operation unit 161 with respect to the time frame in which the examination can be reserved from the time table displayed on the monitor.

In re-designing for change in reservation, the candidate of the time frame in which the reservation can be changed is displayed on the monitor of the display unit 162 through the reservation change candidate presenting unit 156, where the change of the examination reservation with respect to the time frame in which the reservation is already made can be carried out using the user interface such as the mouse connected to the operation unit 161.

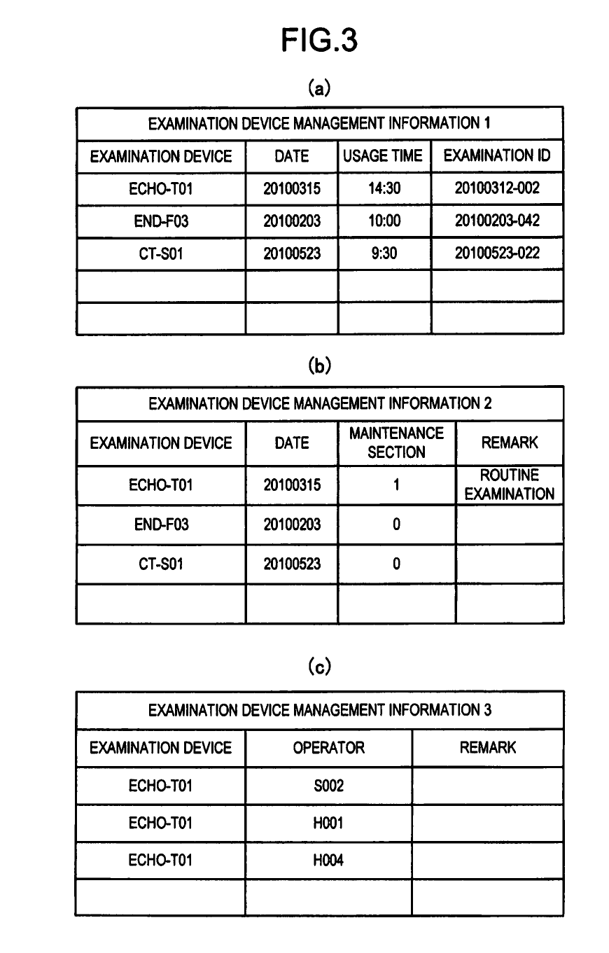

The details of the database table saved in each database will now be described. A basic table example saved in the examination device database 11 is shown in FIG. 3. FIG. 3(a) shows a relationship of the examination device and the examination ID for the examination device management information 1. The date to use, the usage time, the corresponding examination ID, and the like are managed for every examination device. In the following description, the time frame will be described in units of 30 minutes regardless of the type of examination to facilitate the explanation, but the reservation management may be carried out in any time unit. As the standard examination time generally differs depending on the type of examination, an efficient and fine reservation management can be carried out by carrying out the reservation management with a collection of a plurality of time frames of about 15 minutes as a reservation unit according to the type of examination.

FIG. 3(b) shows a relationship example of the examination device and the maintenance information for the examination device management information 2. The maintenance information such as the periodic maintenance, the failure, and the like are managed for the examination device possessed in the hospital. The maintenance section is prepared for each examination device, so that use and reservation cannot be made on the maintenance day. In this example, the maintenance section of one day unit is set, but the maintenance section may be set in time unit.

FIG. 3(c) shows a relationship example of the examination device and the operator for the examination device management information 3. A list of operators permitted to operate is saved with respect to each examination device.

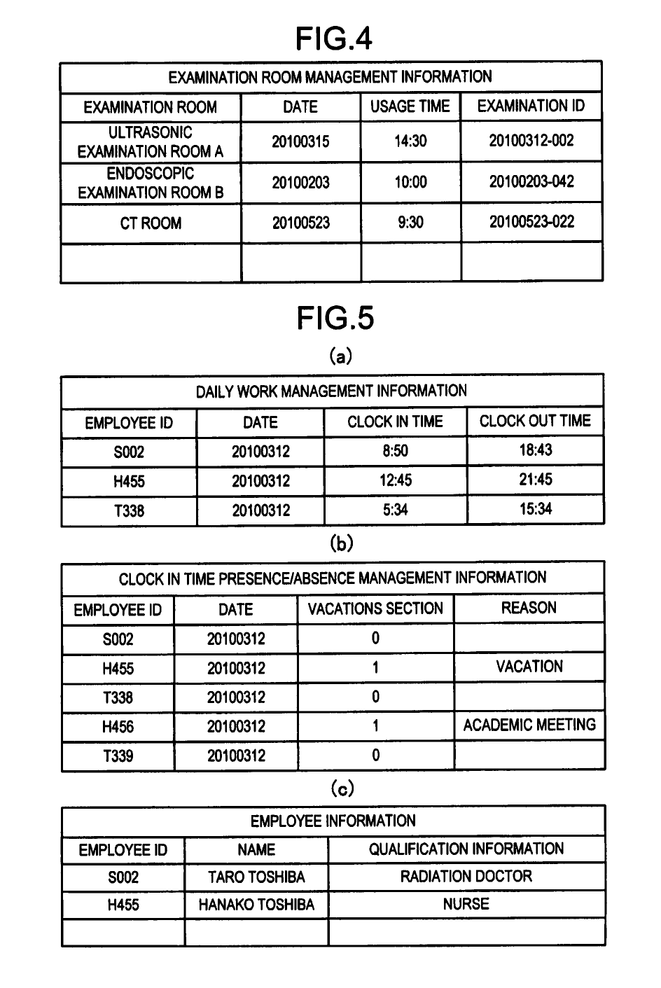

FIG. 4 is a view showing a basic table example saved in the examination room database 12. The date to use, the time, and the corresponding examination ID are managed with respect to each examination room. The examination device used in each examination room can be managed since the examination device to use is associated with respect to the examination ID.

FIG. 5 is a view showing a basic table example saved in the work management database 13. FIG. 5(a) sows the daily work management information of the current day, where the employee ID, date, clock in time, clock out time, and the like are managed. The daily work management information is automatically acquired and saved by a time card or the like possessed by each employee.

FIG. 5(b) is a view showing an example of clock in time presence/absence management information. This shows the schedule such as the vacation schedule of each employee, where the employee ID, date, vacation section, reason, and the like are managed. In this example, the vacation section of one day unit is set, but the vacation section may be set in time unit.

FIG. 5(c) is a view showing an example of employee information. The examination by a licensed employee permitted to operate the examination device such as doctors, examination technicians, or nurses is carried out by managing the employee ID, name, qualification, and the like.

In addition to the name of the disease of the patient, the site to be examined, and the type of examination device as described above, the patient information database 14 saves and manages the priority information of the examination reservation with the examination ID using the in/out section indicating whether an admitted patient or an outpatient, the emergency section representing the degree of emergency of the patient, and a changing section representing the changing level of reservation.

The emergency section is sectionalized into two large sections, emergency and normal, where the emergency level can be set in the lower section if the emergency section is emergency. For instance, the emergency level can be set according to the conscious level of the patient as an example of the emergency level. The conscious level evaluation such as JCS (Japan Come Scale) or GCS (Glasgow Come Scale) may be used or a unique conscious level in the hospital may be set as an example of the conscious level of the patient.

For instance, in the example of the JCS, the level is greatly divided into three stages and each stage is further categorized into three to thus represent the conscious level of a total of 9 stages, where the consciousness disturbance is more severe the greater the numerical value.

(1) The conscious level of waking without stimulation is sectionalized into three stages of (a) state of having substantially clear conscious but not quite distinct, (b) state of having impairment in orientation (recognition of time, place, person), and (c) state of not being able to say his/her name and birthday.

(2) The conscious level of the state of waking when stimulated (sleep when stimulation is stopped) is sectionalized into three stages of (a) state of opening the eyes when normally called, responding to instructions such as “grip right hand”, words can be spoken although with many mistakes, (b) state of opening the eyes when called loudly or when the body is shook, and (c) state of opening the eyes slightly when called without providing pain stimulation, responding to simple instructions such as “grip hand”.

(3) The conscious level of not waking even if stimulated is sectionalized into three stages of (a) state of performing the operation of brushing aside with respect to the pain stimulation, (b) state of slightly moving hands and legs or making a frown face with a pain stimulation, and (c) stage of not responding to the pain stimulation.

With respect to the changing section, a plurality of levels can be set for the changing level of reservation. For instance, the changing levels such as (1) changeable, (2) unchangeable, (3) only raising is possible, and (4) order holding change can be set.

The changeable level is a state in which the reservation can be freely changed afterwards with respect to the interruption reservation of another patient. In particular, setting with respect to the outpatient waiting for his/her turn is suitable.

The unchangeable level is a state in which the reservation cannot be changed with respect to the interruption reservation of another patient. The unchangeable level is set in a case where problems arise when change is carried out such as in the case of pre-operative examination or in the case of VIP patient.

The only raising is possible level is set when reservation can be changed only in raising when the examination is cancelled.

The order holding change level is set when a plurality of examinations is set in a series of schedules, and interchanging of the examination order is prohibited. With such setting, the reserved time frame can be changed so that the examination order does not change.

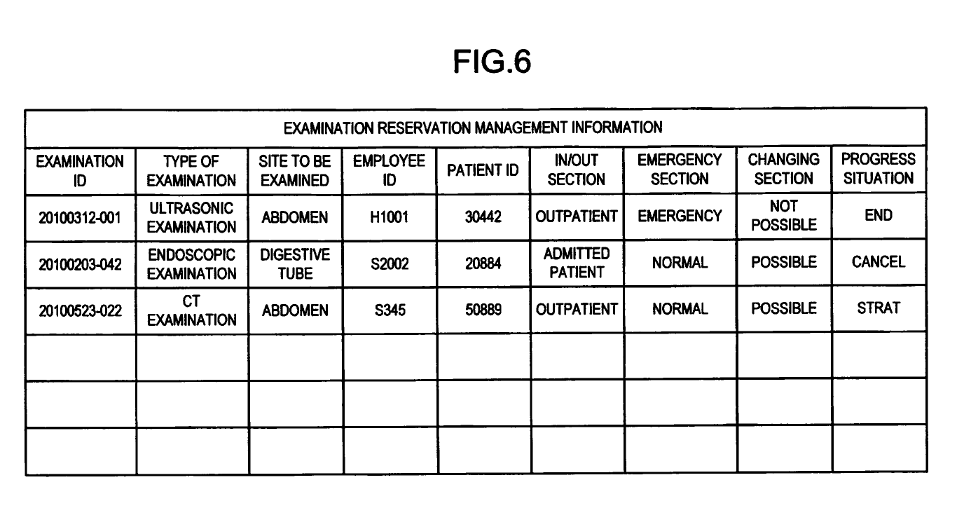

FIG. 6 is a view showing a basic table saved in the reservation management database 151. The type of examination, the examination site, the employee ID of the operator, the patient ID, the in/out section, the emergency section, the changing section, the progress situation, and the like are managed with respect to each examination ID. The progress situation is real time information with respect to the examination, and includes statuses such as start, end, and cancel.

When carrying out the examination reservation, the relationship of each database will be described. First, the examination room, the examination device and the like corresponding to the type of examination are searched with respect to the type of examination such as ultrasonic wave, endoscopic examination, and CT examination. For instance, the ultrasonic examination room A is searched by the examination room management information of FIG. 4 for the ultrasonic examination.

At the same time, an ultrasonic diagnosis device ECHO-T01 is searched by the examination device management information of FIG. 3(a) for the device necessary for the ultrasonic examination. In this case, the maintenance information of the ultrasonic diagnosis device ECHO-T01 is checked by the examination device management information 2 of FIG. 3(b). If the ultrasonic diagnosis device ECHO-T01 is usable, the operator who can operate the ultrasonic diagnosis device ECHP-T01 is supposedly assigned as an operator 5002 by the examination management information 3 of FIG. 3(c). With respect to the operator 5002, the name and qualification are searched by the employee information of FIG. 5(c), and at the same time, the daily work management information of FIG. 5(a) and the clock in time presence/absence information is searched. If there is no problem, the operator is officially assigned, the examination ID is issued, and the reservation is completed.

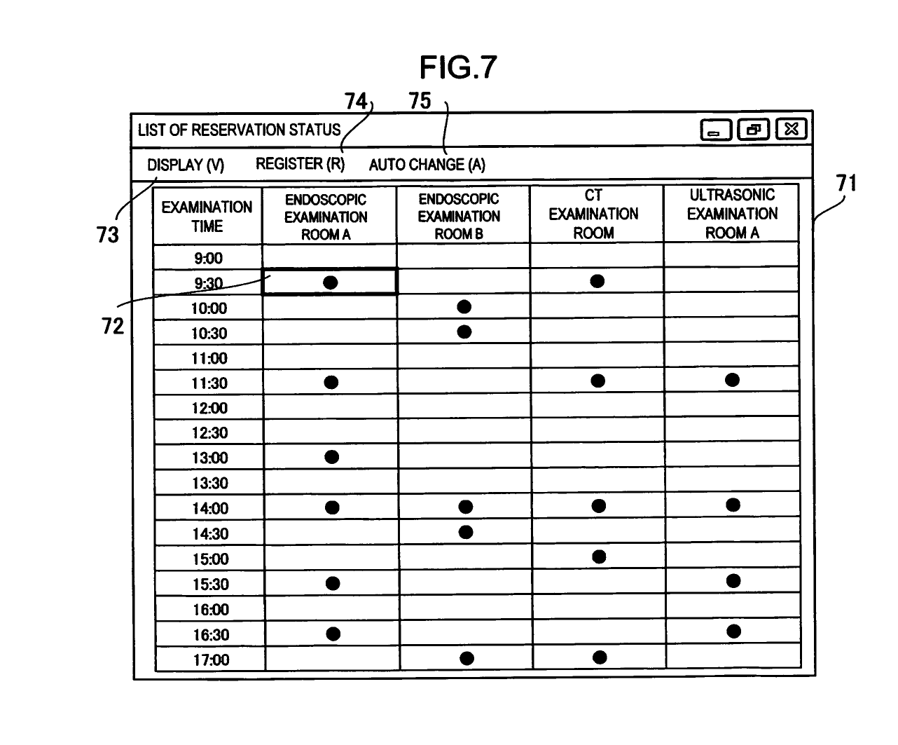

FIG. 7 is a view showing a list of reservation status of the examination reservation management system. The reservation list is displayed on the display unit 162 of the reservation terminal 16 based on the output of the reservation status presenting unit 154.

For example, a reservation status list 71 of the reservation management system represents time on the vertical axis and the examination room on the horizontal axis. The frame determined from the time and the examination room is referred to as the time frame. In the normal display, the reserved time frame is distinguished with a black circle, or the like. In a specific example, the time frame 72 surrounded with a thick frame shows that the endoscopic examination room A is reserved from 9:30 to 10:00.

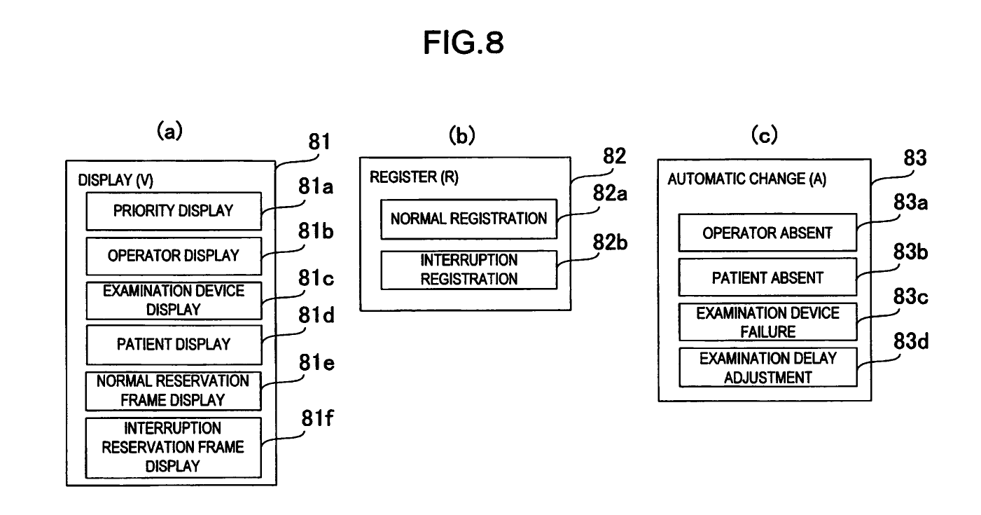

When a display menu 73 of the reservation status list 71 is clicked using a user interface such as a mouse connected to the operation unit 161 of the reservation terminal 16, a display submenu 81 shown in FIG. 8(a) is displayed.

The display submenu 81 includes a priority display 81a, an operator display 81b, an examination device display 81c, a patient display 81d, a normal reservation frame display 81e, and an interruption reservation frame display 81f.

The priority display 81a displays priority with respect to each reserved time frame of the reservation status list 71. The display method may be displaying a numerical value of the priority with respect to the reserved time frame, or may be displaying in a color-coded manner such as “red”, “yellow”, and “blue” according to the priority.

The operator display 81b displays the name of the operator charge of the relevant time frame in the selected time frame 72. The time frame handled by the same operator may be displayed in an emphasized manner.

The examination device display 81c displays the examination device used in the relevant time frame in the selected time frame 72.

The patient display 81d displays the name of the patient to be subjected to the examination in the relevant time frame in the selected time frame 72.

The normal reservation frame display 81e displays in an emphasized manner such as by color coding the time frame that is not yet reserved and can be reserved in the reservation status list 71. In particular, the time frame closest to the present may be presented in an emphasized manner so as to be easily recognized by a person operating the reservation terminal 16.

The interruption reservation display 81f displays in an emphasized manner such as color coding the time frame that can be interrupted. In particular, the time frame closest to the present may be displayed.

<Reservation Registration Method>

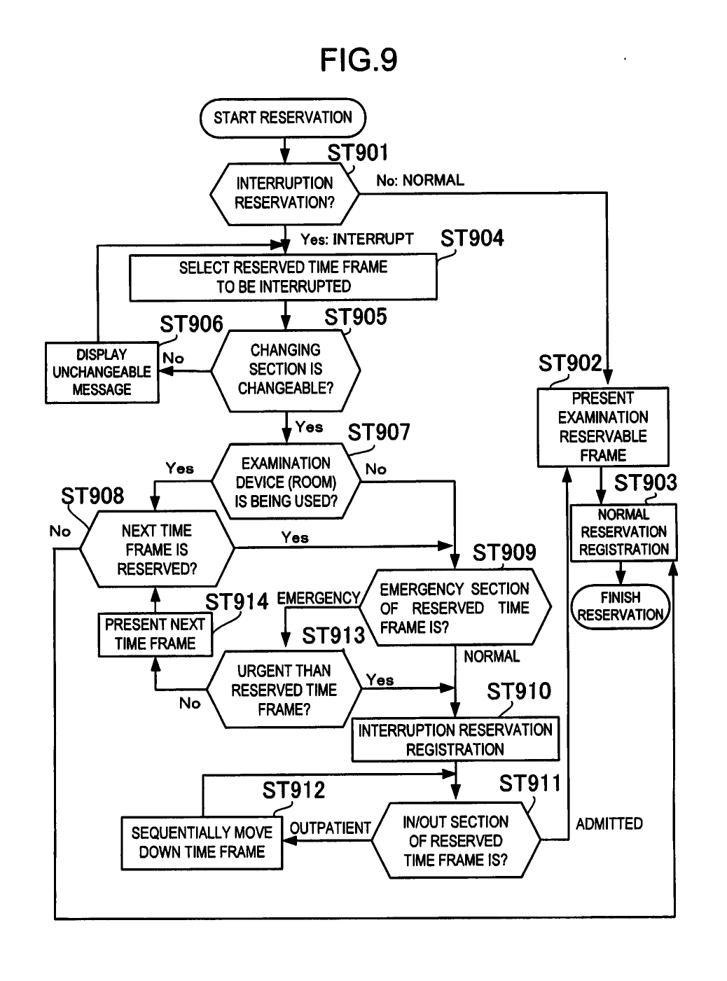

FIG. 9 is a flowchart showing a method of examination reservation registration carried out in new reservation, reservation change, and the like. In the examination reservation registration, the registration can be made by clicking a register menu 74 of the reservation status list of FIG. 7.

As shown in FIG. 8(b), a register submenu 82 includes a normal registration 82a and an interruption registration 82b. The normal registration 82a makes a registration with respect to the time frame that can be reserved, and the interruption registration 82b makes an interruption reservation with respect to the reserved time frame.

In step ST901, determination is first made on whether or not the normal reservation of reserving a time frame in which the examination reservation is not made or an interruption reservation of making an interruption reservation with respect to the reserved time frame. This may be determined by the register submenu 82 selected in the register submenu.

In the case of normal reservation (step ST901: No), the time frame that can be examination reserved is presented (step ST902).

In step ST903, the desired time frame is selected with respect to the presented time frame to confirm the normal reservation registration. In confirming, the reservation is reflected on the reservation management database 151, and at the same time, notification is made that reservation has been made with respect to the examination device database 11, the examination room database 12, the work management database 13, and the patient information database 14.

In the case of interruption reservation (step ST901: Yes), in step ST904, a message informing to select the reserved time frame to be interrupted is displayed on the display unit 162 of the reservation termination 16 with respect to the operator so that the operator of the reservation terminal 16 can select the time frame to be interrupted.

After the reservation frame to be interrupted is selected, the changing section associated with the relevant reservation frame is referenced to determine whether or not change can be made (step ST905). If interruption of the examination reservation is not possible with respect to the selected reservation frame (step ST905: No), a message that interruption is not possible is displayed on the display unit 162 of the reservation terminal 16 (step ST0906), and the reserved time frame to be interrupted, which is different from the previous time, is again selected. In this case, the time frame that can be interrupted may be displayed.

If the interruption of the examination reservation is possible with respect to the selected reservation frame (step ST905: Yes), in particular, if the time frame to be interrupted is the current time, the determination of step ST 907 is made. In step ST907, determination is made on whether or not the examination room is currently being used or the examination device is being used.

This information may be acquired from the progress situation of the examination reservation management information shown in FIG. 6. This corresponds to when the examination cannot be started for some reasons, when cancellation measure is taken, or when the previous examination is prolonged and still not finished at the present time although the current time frame is reserved.

If the examination room is currently being user or the examination device is being used (step ST907: Yes), the interruption cannot be made with respect to the time frame, and thus the next time frame is presented (step ST908). If the next time frame is not reserved (step ST908: No), the normal reservation registration of step ST903 is carried out.

If the next time frame is reserved (step ST908: Yes) or if the examination room is not currently being used (or examination device is not being used) although reservation is made (step ST907: No), the emergency section associated with the time frame to be interrupted is further referenced (step ST909).

If the emergency section of the time frame to be interrupted is normal (step ST909: normal), the interruption can be made and thus the interruption registration is completed (step ST910). If the emergency section of the time frame to be interrupted is emergency (step ST909: emergency), determination is made on which is more urgent in step ST913. The degree of emergency is determined at the emergency level such as the conscious level.

In the case of the patient who urgently requires an examination by the emergency section of the time frame to be interrupted (step ST913: Yes), the interruption registration is carried out.

In the case of the patient who is not urgent by the emergency section of the time frame to be interrupted (step ST913: No), the next time frame is presented (step ST914), and the steps after step ST908 are repeated to register the reservation time.

According to step ST910, the interrupted time frame needs to be changed to another time frame. First, in step ST911, whether the in/out section is the admitted patient or the outpatient is determined.

In the case of the admitted patient (step ST911: admitted), the time frame in which the examination can be reserved is presented (step ST902), and the normal reservation registration is carried out (step ST903). In the case of the outpatient (step ST911: outpatient), the reserved time frame is sequentially brought down for only the patient whose in/out section is outpatient section (step ST902).

As described above, the candidate of the time frame that can be reserved can be presented when carrying out reservation registration or change. The optimum reservation management can be carried out by using the priority information of the patient associated with the time frame. Thus, the examination room, the examination device, and the operator can be assigned on a case by case basis according to the emergency level of the patient, and a very efficient operation can be carried out. Furthermore, the waiting time of the patient can be reduced.

The function in which the examination reservation can be automatically changed will be described below. This function is a menu corresponding to the sudden schedule change. In the automatic change, an automatic change submenu 83 is displayed by clicking an automatic change menu 75 of the reservation status list of FIG. 7, so that automatic change can be made.

As shown in FIG. 8(C), the automatic change submenu 83 includes an operator absence 83a, a patient absence 83b, an examination device failure 83c, and an examination delay adjustment 84d. Such automatic adjustment is carried out by pushing each menu button.

<When Operator is Absent>

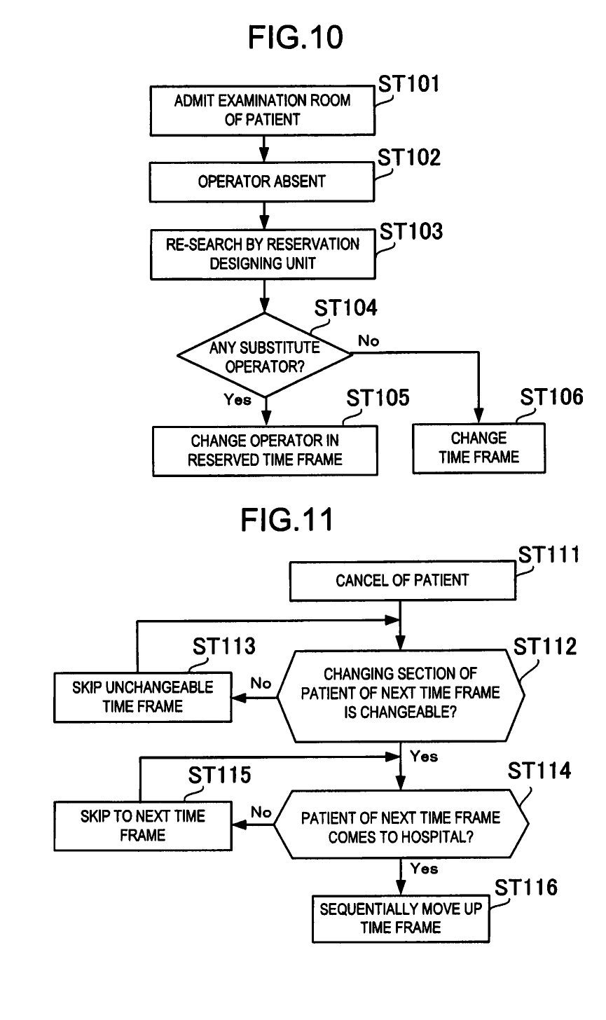

FIG. 10 is a view showing a flowchart of the reservation automatic change of the operator absence 83a. It is a flowchart corresponding to a case in which the operator scheduled to operate the examination device is absent due to an emergency. If the operator suddenly becomes absent (step ST102) although the patient finished admission of the examination room in step ST101, the automatic change menu 75 of FIG. 7, and furthermore, the submenu of the operator absence 83a are selected by the operation unit of the reservation terminal 16. In the reservation designing unit 153, whether or not a substitute operator is present is searched by referencing the examination device database 11 and the work management database 13.

If there is a substitute operator in step ST104 (ST104: Yes), the substitute operator is assigned to the relevant reservation time frame (step ST105). If there is no substitute operator in step ST104 (ST104: No), the time frame is changed. The manner of changing the time frame follows the changing procedure according to the method of FIG. 9 (step ST106).

Thus, and efficient real time operation can be carried out since the substitute operator can be searched even when the operator of the examination device suddenly becomes absent.

<When Patient is Absent>

The reservation automatic change in the case of the patient absence 83b will now be described. FIG. 11 is a view showing a flowchart of the reservation automatic change of when the patient is absent. When referring to the patient being absent, this is when the outpatient cannot go to the hospital due to an emergency and when the admitted patient dies unfortunately. The automatic change menu 75 of FIG. 7 and the submenu of the patient absence 83b are selected by the operation unit of the reservation terminal 16.

In step ST111, the cancel information of the examination reservation of the patient is acquired. In step ST112, the changing section of the patient associated with the next time frame is determined, where if the changing section is unchangeable (ST112: No), this time frame is skipped and the determination is similarly made on the changing section of the patient for the next time frame. If the changing section is changeable (ST112: Yes), determination is made on whether or not the patient of the next time frame has come to the hospital (or admitted) in step ST114.

If the patient of the next time frame has not come to the hospital (or admitted) (ST115: No), determination is further made similarly on whether or not the patient has come to the hospital (or admitted) for the next time frame.

If the patient of the next time frame has come to the hospital (or admitted) (step ST115: Yes), the time frame is sequentially moved up (step ST116).

Therefore, when the patient suddenly becomes absent, the time can be moved up so that the waiting time of the patient can be reduced and an efficient real time operation can be carried out.

<When Examination Device Breaks Down>

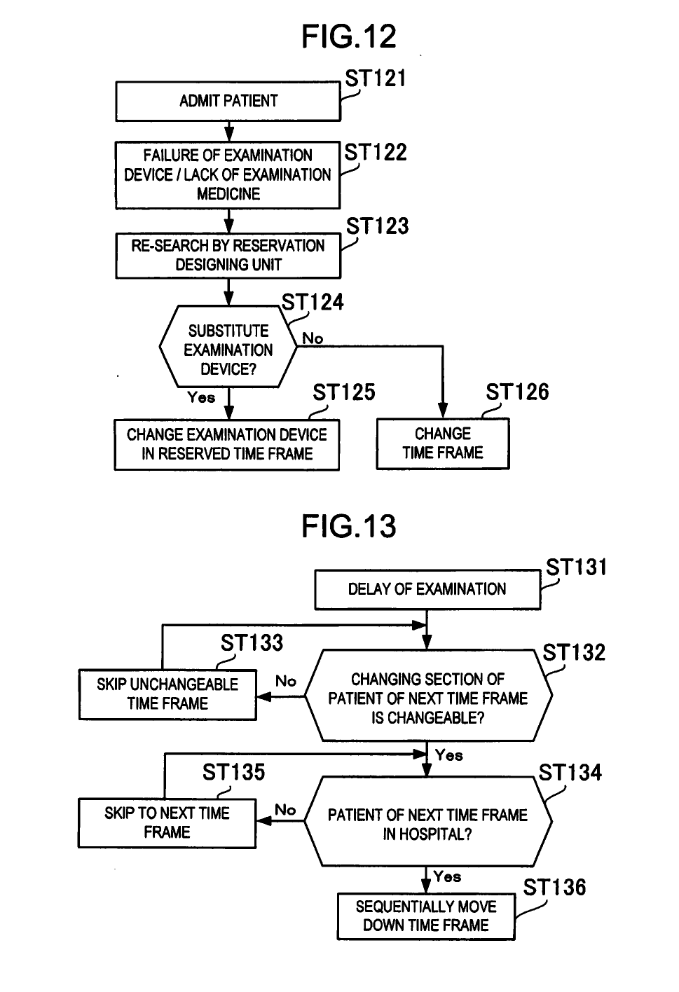

FIG. 12 is a view showing a flowchart of the reservation automatic change for the case of the examination device failure 83c. It is a flowchart corresponding to when the examination device suddenly breaks down or when the stock of examination medicines and the like used in the device runs out and examination cannot be carried out. The configuration to respond to the case where the stock of the examination medicines and the like runs out is described in the second embodiment.

If the examination cannot be carried out all on a sudden in step ST122 although the patient finished admission of the examination in step ST121, the automatic change menu 75 of FIG. 7 is selected and the submenu of the examination device failure 83c is selected by the operation unit of the reservation terminal 16. In the schedule designing unit 153, whether or not there is a substitute device and an examination room is searched by referencing the examination device database 11 and the examination room database 12.

If there is a substitute examination device in step ST124 (step ST124: Yes), the substitute examination device is assigned to the relevant reservation time frame (step ST125). If there is no substitute examination device in step ST124 (step ST104: No), the time frame is changed. The manner of changing the time frame follows the changing procedure according to the method of FIG. 9 (step ST106).

Thus, the efficient and real time operation can be carried out since the substitute examination device can be searched even when the examination device suddenly breaks down.

<In examination Delay Adjustment>

FIG. 13 shows a flowchart of the reservation automatic change when the examination delay adjustment 84d occurred.

The examination delay is when the progress of the examination is not transitioning as the examination reservation. In such a case, there is a need to move down the order of examination and perform time adjustment. In this case, the automatic change menu 75 of FIG. 7, and furthermore, the submenu of the examination delay adjustment 83d are selected by the operation unit of the reservation terminal 16.

In step ST131, the delay information of the examination is acquired. In step ST132, the changing section of the patient associated with the next time frame is determined, where if the changing section is unchangeable (step ST132: No), the time frame is skipped and the determination is made on the changing section of the patient similarly for the next time frame. If the changing section is changeable (step ST132: Yes), whether the patient of the next time frame has come to the hospital (or admitted) is determined in step ST134.

If the patient of the next time frame has not come to the hospital (or admitted) (step ST135: No), whether or not the patient has come to the hospital (or admitted) similarly determined for the next time frame.

If the patient of the next time frame has come to the hospital (or admitted) (step ST135: Yes), the time frame is sequentially moved down (step ST136).

Therefore, when there is a delay in the examination, the time is automatically is moved down thus enabling an efficient operation.

As described above, according to the first embodiment, the presentation of the time frame and the automatic recombination of the time frame are carried out using the priority information, so that a reservation management system enabling an efficient operation can be provided.

Furthermore, the problem in that the device can be reserved even if the operator of the examination device has not come in and the examination cannot be performed, and the problem in that the examination can be reserved even if the examination device is being maintenance or is broken down without being associated with such information do not arise.

In the case of a plan where the patient is to be continuously performed with a plurality of examinations, even if there is a delay in a certain examination, the examination can be carried out without changing the order. The waiting time of other patients can be reduced since the examination device or the examination room does not uselessly become available.

Moreover, a case where the reserved examination is not performed in the relevant time band and the examination device is not used such as when the scheduled patient does not come to the hospital can be prevented.

### Second Embodiment

In addition to the first embodiment, the second embodiment includes a medicine information database 18 on the network 17. The usage amount and the stock of the medicine can be easily managed in synchronization with the performance or the reservation of the examination.

FIG. 15 is a view showing a basic table example saved in the medicine information database 18. As shown in FIG. 15(a), a list of necessary medicines is shown with respect to the type of examination, and the amount used in one examination is managed. As shown in FIG. 15(b), the stock amount (remaining amount) of each medicine at the time the examination reservation is completed is managed.

According to the second embodiment, lack of examination medicine does not occur since each medicine is automatically managed in synchronization with the examination reservation in addition to the effect of the first embodiment. Therefore, the check for lack of examination medicine in step ST122 of FIG. 12 is not necessary.

The present invention is not limited to the above embodiments, and various modifications can be made. The time of the time frame is the same regardless of the type of examination in the embodiment described above, but this is for the sake of convenience of explanation, and a plurality of reservation frames and one reservation frame can be interchanged in the type of examination where the examination time differs. Furthermore, the patients can be interchanged in the reservation frame and the reservation frames can be interchanged.

Some of the embodiments of the present invention have been described, but such embodiments are presented as examples and are not intended to limit the scope of the invention. The novel embodiments can be implemented in various other modes, where various omissions, replacements, and changes can be made within a scope not deviating from the gist of the invention. Such embodiments and variants are within the scope and gist of the invention, and are within a scope equivalent to the invention described in the Claims.

### REFERENCE SIGNS LIST

11 Examination device database
12 Examination room database
13 Work management database
14 Patient information database
15 Reservation management server
16 Reservation terminal
17 Network
18 Medicine information database
151 Reservation management database
152 Information acquiring unit
153 Reservation designing unit
154 Reservation status presenting unit
155 Priority information determining unit
156 Reservation change candidate presenting unit
161 Operation unit
162 Display unit

1. An examination reservation management system comprising:
an information acquiring means for acquiring examination reservation information including priority information indicating priority of an examination;
a reservation status presenting means for distinguishing and displaying a time frame in which examination can be reserved and a reserved time frame based on the examination reservation information;
a reservation input means for making a new examination reservation;
a priority information determining means for determining whether or not the new examination reservation can be reserved using the priority information; and
a reservation designing means for confirming the examination reservation from the determination result by the priority information determining means.

2. The examination reservation management system according to claim 1, wherein the examination priority information includes at least one of an emergency section indicating a degree of emergency of a patient, an in/out section indicating admitted patient or outpatient, and a changing section indicating necessity of change of the examination reservation.

3. The examination reservation management system according to claim 2, wherein the reservation status presenting means displays the time frame in a form of a time table, and displays the examination reservation information in a distinguished manner.

4. The examination reservation management system according to claim 3, wherein the examination reservation information further includes at least one of patient information to be examined, examination device information including operation information of an examination device, an examination room information including an operation status of the examination room, and work information of an operator of the examination device.

5. The examination reservation management system according to claim 4, wherein the examination device information further includes a maintenance section indicating maintenance information of the device.

6. The examination reservation management system according to claim 5, wherein the work information of the operator includes work status of current day and work plan information after current day.

7. The examination reservation management system according to claim 6, further comprising a means for automatically changing the reservation of the patient, the operator, the examination device, and the examination room based on the examination reservation information.

8. The examination reservation management system according to claim 7, wherein the information acquiring means acquires medicine information indicating type and usage amount of a medicine used in the examination, and carries out stock management of the medicine in synchronization with the examination reservation information.

9. The examination reservation management system according to claim 8, wherein a conscious level of the patient is used for the emergency section of the patient.

10. An examination reservation management system comprising:
an information acquiring means for acquiring examination reservation information including priority information indicating priority of an examination;
a reservation status presenting means for distinguishing and displaying a time frame in which examination can be reserved and a reserved time frame based on the examination reservation information;
a reservation input means for making an interruption of examination reservation with respect to the reserved time frame;
a means for associating the examination reservation information with respect to the reserved time frame;
a priority information determining means for determining whether or not the examination reservation change can be carried out using the examination reservation information; and
a reservation designing means for confirming the examination reservation from the determination result by the priority information determining means.

11. The examination reservation management system according to claim 10, wherein the examination priority information includes at least one of an emergency section indicating a degree of emergency of a patient, an in/out section indicating admitted patient or outpatient, and a changing section indicating necessity of change of the examination reservation.

12. The examination reservation management system according to claim 11, wherein the reservation status presenting means displays the time frame in a form of a time table, and displays the examination reservation information in a distinguished manner.

13. The examination reservation management system according to claim 12, further comprising a reservation change candidate presenting means for presenting a candidate of a time frame in which reservation is changeable based on the examination reservation information.

14. The examination reservation management system according to claim 13, wherein the examination reservation information further includes at least one of patient information to be examined, examination device information including operation information of an examination device, examination room information including an operation status of the examination room, and work information of an operator of the examination device.

15. The examination reservation management system according to claim 14, wherein the examination device information further includes a maintenance section indicating maintenance information of the device.

16. The examination reservation management system according to claim 15, wherein the work information of the operator includes work status of current day and work plan information after current day.

17. The examination reservation management system according to claim 16, further comprising an automatic changing means for automatically changing the reservation of the patient, the operator, the examination device, and the examination room based on the examination reservation information.

18. The examination reservation management system according to claim 17, wherein the information acquiring means acquires medicine information indicating type and usage amount of a medicine used in the examination, and carries out stock management of the medicine in synchronization with the examination reservation information.

19. The examination reservation management system according to claim 18, wherein a conscious level of the patient is used for the emergency section of the patient.

20. The examination reservation management system according to claim 19, wherein the automatic changing means is carried out in real time based on a progress situation of the examination.
